(19) United States
(12) Patent Application Publication
(10) Pub. No.: US 2024/0404665 A1
(43) Pub. Date: Dec. 5, 2024

(54) Vaccination Status Identification Card

(71) Applicant: Tonya Smith, Sunrise, FL (US)

(72) Inventor: Tonya Smith, Sunrise, FL (US)

(21) Appl. No.: 18/800,931

(22) Filed: Aug. 12, 2024

Related U.S. Application Data
(63) Continuation-in-part of application No. 17/855,296, filed on Jun. 30, 2022.

(51) Int. Cl. G16H 10/65; G16H 20/00

(52) U.S. Cl. CPC G16H 10/65; G16H 20/00

(57) ABSTRACT

A method, a system and a computer program product track vaccinations received by an individual and provides proof of the individual's vaccination status. A vaccine status identification (VSID) module/utility establishes a vaccination schedule for administering a specified vaccine via one or more vaccine injections/shots to an individual. The VSID module determines whether the individual receives one or more vaccine injections for the specified vaccine according to the vaccination schedule. The VSID module determines a vaccine status of the individual based on received vaccine injections and the vaccination schedule. The VSID module integrates information associated with vaccination status and the vaccination schedule into a secure readable/scannable vaccine identification card issuable to the individual. The VSID module provides secure access to information on the readable vaccine ID card and provides an alert each time a unique code corresponding to the vaccine ID card is utilized.

### BACKGROUND OF THE INVENTION

This invention relates generally to a tracking and identification system. This invention relates more particularly to an apparatus/device for embedding tracking software into various forms of identification. This invention relates generally to apparatuses and devices for tracking and identifying individuals and objects. This method also can be used with various forms of digital and physical IDs. This invention relates more particularly to a system to enhance safety and security through real-time tracking and reporting.

As COVID-19 vaccines became commonly available nationwide, many employers began either requiring or strongly encouraging their employees to get vaccine shots. These employers and their organizations/corporations are hoping to leverage widespread vaccinations to quickly return to pre-pandemic business as usual and to limit workplace infections that could once again temporarily shut down business operations. Proof of COVID-19 vaccination or a negative COVID-19 result from a screening test could be a way for businesses and schools to provide continuing safety mechanisms and to reassure customers, students, and parents that steps are being taken to limit transmission of the virus.

Currently, a one-size fits all approach is being applied where individuals are given the same access to venues as long as they wear a mask. However, vaccine status is rarely used to control or limit access to places/venues and/or to determine mask wearing requirements. Those who have decided to abide by CDC recommendations by obtaining a COVID-19 vaccination are frustrated with the lack of reasonable allowances/privileges that vaccination was expected to provide. Furthermore, many individuals are worried about the risk of fraud associated with vaccine data and status.

The Problem: There is a need for efficient systems to track individuals who have been exposed to infectious diseases. This is crucial for controlling the spread of illnesses and ensuring timely medical intervention. Law enforcement requires better methods to track suspects of crimes, especially in cases of abduction or other serious offenses, to ensure public safety and facilitate quicker resolution of investigations. Locating missing persons, such as Amber Alert victims, is a critical need for families and authorities to reunite lost individuals with their loved ones promptly. There is a demand for effective tracking and recovery systems for stolen vehicles to aid in reducing crime rates and recovering property.

The Solution: Embedded Software in IDs: By embedding tracking and reporting software into various forms of identification, such as digital IDs, government IDs, travel IDs, and state registrations (e.g., cruise onboard cards, driver's licenses, passports, and license plates), the system can leverage existing ID infrastructure for new functionalities. Connected to Hosted Software: These IDs will be connected to a centralized hosted software platform, which will manage the data and operations, ensuring seamless integration and real-time updates.

Manufacturing Process: Access Via QR Code or Chip: The tracking device will be embedded in physical ID cards through a QR code or a microchip, making it easy to use and integrate into existing ID formats. Device as a Byproduct of Hosted Software: The software's primary functionality will generate the tracking capabilities as a byproduct, ensuring it remains cost-effective and efficient. Manufactured in Private Facilities: Initially, the devices will be produced in private facilities with the capability to scale production as needed. State Regulatory Office Production: With appropriate licensing, state regulatory offices can also manufacture and issue these devices, ensuring widespread availability. Data Access Restrictions: External access to the data will be restricted unless mandated by legal requirements, ensuring user privacy and data security. User and Household Permission: Certain features of the device will only activate with explicit permission from the user and, where applicable, household members, ensuring consent and control over personal data.

Operation in Specific Scenarios: Criminal Cases and Missing Persons: If a user is a suspect in a criminal case, such as child abduction, or if a person is reported missing, an alert will be issued. The prerecorded information in the database allows for immediate activation of a tracking system upon request from authorized parties (household members or government entities). This will enable the precise location of the individual to be provided to the local police department for swift action. Travel and Infectious Diseases: For travel scenarios, each member of a household will receive a Travel ID specific to the travel vehicle (e.g., cruise ship, tourist group). If a user becomes ill with an infectious disease, household members and other travelers can report exposure and symptoms to the centralized database. The software will trace the origin of exposure, alert individuals who may have been at risk, and suggest remedies for known illnesses. This system helps in managing outbreaks and preventing further spread of diseases.

The Innovation: Quick and Accurate Location of Abducted Individuals: The embedded ID allows for rapid and precise location tracking of abducted individuals, enhancing the chances of their safe recovery. Enhanced Disease Management on Cruise Ships: In cases of infectious disease outbreaks on cruise ships, the early detection and reporting system provide the crew with ample time to implement isolation measures, thereby reducing the spread of the infection among passengers. Autonomous Data Processing: The software algorithm will autonomously trace locations, issue alerts, study movement patterns, track disease outbreaks, suggest remedies, and limit exposure. By collecting basic user information, it enhances the overall safety and health of individuals through proactive measures and informed decision-making.

### BRIEF SUMMARY OF THE INVENTION

The present invention is directed to a method, a system and a computer program product that tracks vaccinations received by an individual and provides proof of the individual's vaccination status. A vaccine status identification (VSID) module/utility establishes a vaccination schedule for administering a specified vaccine via one or more vaccine injections/shots to an individual. The VSID module determines whether the individual receives one or more vaccine injections for the specified vaccine according to the vaccination schedule. The VSID module determines a vaccine status of the individual based on received vaccine injections and the vaccination schedule. The VSID module integrates information associated with vaccination status and the vaccination schedule into a secure readable/scannable vaccine identification card issuable to the individual. The VSID module provides secure access to information on the readable vaccine ID card and provides an alert each time a unique code corresponding to the vaccine ID card is utilized.

According to an aspect, the vaccination card is a COVID-19 Identity (C-ID) card/document. According to one or more related aspects, the vaccination/C-ID card is a standalone plastic card that is carried by the individual to enable fast efficient access to vaccine status data used to limit/control access to specific locations.

According to an aspect, the C-ID card or corresponding information is integrated with and/or linked to a government issued ID such as a driver's license or a state ID card.

According to another aspect, the C-ID card/document can be utilized within a fast pass mechanism/system. For example, the VSID module provides secure fast access/entrance to individuals traveling through airports, cruise ships, and large events, such as professional, sporting or entertainment events held within stadiums. According to an aspect, the VSID module provides an additional layer of security to a safety screening process.

According to an aspect, COVID-19 vaccine data is uploaded onto the scannable card. According to the aspect, the scannable card is designed to support access to vaccine data using one or more of a QR code and a bar code.

According to an aspect, the VSID module provides secure access to vaccine data via a smart-phone App.

According to an aspect, the VSID module is able to cross reference data with a government's Center for Disease Control and Prevention “CDC” database to prevent unauthorized data duplication.

According to an aspect, the VSID module is able to update the card holder's vaccine information within specified time periods based on a schedule that must be followed in order to maintain a partial or complete vaccination status, according to vaccination guidelines and/or an established vaccination schedule.

According to one or more aspects, the VSID module provides the individual with an alert each time someone uses the individual's unique app code. According to one or more related aspects, the VSID module provides the alert as a fraud alert text.

According to one or more aspects, the VSID module enables an individual to establish, using a smart-phone App, a group such as a household group for travel purposes, by which a single QR code can be used to access vaccination data for all persons within the established group.

According to an aspect, the VSID module enables individuals that obtain the App to join or add themselves to the household group. According to an aspect, the VSD module may require that the individual submitting a request to be added to the household group use a verification code to confirm the request to be added to the household group.

According to one or more related aspects, the VSID module enables the household group to receive access to travel related services/products at discounted/group rates.

According to one or more aspects, the VSID module can specify a limit on the number of individual members in a group and a number of associated groups per App. For example, the VSID module may specify a limit of eight (8) members in a group and a maximum of two associated groups per App. Thus, a maximum number of sixteen (16) members from a household can utilize a single App when split into two associated groups, each having a maximum of 8 members.

According to a first aspect of the present invention, there is a method for providing a proof and a use of a first user's vaccination status comprising: (A) establishing a vaccination schedule and a date deadline for an administering of a vaccine to said first user; (B) verifying whether said first user has received said vaccine on or before said date deadline according to said vaccination schedule by a collection of a vaccine data from a vaccine provider; (C) determining a vaccine status of said first user from whether said first user has received said vaccine on or before said date deadline according to said vaccination schedule; (D) integrating an information associated with vaccine status and vaccine schedule into a secure readable/scannable vaccine identification card issuable to said first user; (E) providing a secure access to said information on said secure readable/scannable vaccine identification card; and (F) providing an alert each time a unique code corresponding to the vaccine ID card is utilized.

According to a second aspect of the present invention, there is a method for providing a proof and a use of a first user's vaccination status further comprising including said first user in a household group having at least two users comprising a plurality of users including at least said first user and a second user before step (B).

According to a third aspect of the present invention, there is a method for providing a proof and a use of a first user's vaccination status further comprising creating a vaccination data group for said household group and determining a group vaccine status for said household group as part of step (C).

According to a fourth aspect of the present invention, there is a method for providing a proof and a use of a first user's vaccination status further comprising accessing an application and entering a name for each user on a data entry page for users to be added to said household group before step (B).

According to a fifth aspect of the present invention, there is a method for providing a proof and a use of a first user's vaccination status further comprising each user in said plurality of users submitting a request to be added to said household group and using a verification code to confirm said request to be added to said household group.

According to a sixth aspect of the present invention, there is a method for autonomously monitoring user location, health status, and risk of surrounding infections further comprising the provision of basic user information and geographical position. This method involves continuously collecting and analyzing data from various sources, including GPS coordinates to track the user's location and sensors to monitor vital health parameters such as temperature, heart rate, and respiratory rate. The system integrates this data with information on current infection hotspots and reported cases of infectious diseases within the user's vicinity. By comparing the user's health status and location data against the epidemiological data, the system can assess the user's risk of exposure to infections. Additionally, the system securely stores basic user information, such as age, vaccination history, and underlying health conditions, to provide personalized risk assessments and recommendations. This comprehensive approach enables real-time, proactive health management, allowing individuals to take informed actions to protect their health and reduce the spread of infections.

The described method goes significantly beyond mere software by detailing a comprehensive, multi-step process that integrates various practical and technical elements for managing and verifying a user's vaccination and health status. Here is a breakdown of the claims and the processes they encompass:

Establishing a Vaccination Schedule and Deadline: This step involves setting a timeline and specific dates by which the first user needs to receive their vaccination. This is akin to a project management task where specific milestones (in this case, vaccination dates) are defined.

Verifying Vaccination by Collecting Data: This step entails collecting vaccination data from healthcare providers to check if the user has been vaccinated as per the schedule. It involves interfacing with external entities (vaccine providers) to gather necessary data, which may involve secure communication channels and data handling protocols to ensure privacy and accuracy.

Determining Vaccine Status: Based on the collected data, this step determines the vaccination status of the user. It involves comparing the actual vaccination data against the predefined schedule to ascertain compliance. This could involve logic and algorithms to evaluate the data.

Integrating Vaccine Status into a Secure ID Card: The user's vaccination status and schedule information are integrated into a secure, readable, or scannable identification card. This step includes designing and implementing a secure method for embedding and encoding this information into a physical or digital card, which could involve cryptographic techniques to ensure the security of the data.

Providing Secure Access to Information: The method provides a way to securely access the information on the ID card. This could include technologies such as QR codes, NFC (Near Field Communication), or other secure scanning methods that allow authorized personnel to access the information without compromising security.

Alert System for ID Card Usage: Every time the unique code corresponding to the vaccine ID card is used, an alert is generated. This involves setting up a monitoring and alerting system that tracks the usage of the ID card and sends notifications, which may require real-time data processing and communication infrastructure.

Determining Health and Risk Status: Based on the collected data, this step determines the health status of the user as well as surrounding risks. It involves storing symptomatic information or confirmed descriptions of illness and utilizing geographic positioning to determine sources of origin. Additionally, it shares this data with surrounding users to warn them of possible exposure. This involves the creation of automated software, secure servers, and advanced API systems.

Additional claims extend the method's applicability to household groups, adding layers of complexity:

Including Users in a Household Group: Before verifying vaccination data, users can be grouped into household units, allowing for collective management of vaccination data and statuses. This involves creating and managing group data structures, user relationships, and shared information.

Creating a Vaccination Data Group: For household groups, a collective vaccination status is determined. This requires aggregating individual data to generate a group-level status, which could involve statistical methods or rules-based systems.

User Data Entry for Household Groups: Users can access an application to enter names and create household groups. This step necessitates the development of user interfaces, data entry forms, and backend systems to handle and store this information securely.

Verification and Request Management: Users request to be added to household groups, and these requests are verified using unique codes. This step involves implementing request handling workflows, user verification processes, and secure code generation and validation mechanisms.

Overall, this method outlines a detailed, multi-faceted approach that combines aspects of project management, data collection and verification, secure data integration, user interface design, and real-time alerting systems, reflecting a blend of software development, secure data handling, and system integration techniques.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 illustrates a block diagram representation of an example data processing system within which certain features of the present disclosure can be implemented, according to one or more embodiments.

FIG. 2 illustrates a front side of a government-issued vaccination ID card, according to one or more embodiments of the disclosure.

FIG. 3 illustrates a back side of a government-issued vaccination ID card, according to one or more embodiments.

FIG. 4 illustrates a bar-code on a government-issued vaccination ID card being read by a scanner, according to one or more embodiments of the disclosure.

FIG. 5 illustrates a vaccination APP being accessed by reading a QR code on a government-issued vaccination ID card using a smart-phone camera, according to one or more embodiments of the disclosure.

FIG. 6 illustrates a user interface providing a vaccination status of an individual, according to one or more embodiments of the disclosure.

FIG. 7 illustrates a user interface showing a number of selectable menu buttons used to provide vaccine-related information, according to one or more embodiments of the disclosure.

FIG. 8 illustrates a user interface showing a user information page for entry of vaccination data/schedule information, according to one or more embodiments of the disclosure.

FIG. 9 illustrates a user interface providing a household group page for entering names of members to be included in the group, according to one or more embodiments of the disclosure; and

FIG. 10 presents a flow chart illustrating the process of tracking received vaccinations and vaccination status, according to one or more embodiments.

Like reference numerals refer to like parts throughout the several views of the drawings.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations.

All of the implementations described below are exemplary implementations provided to enable persons skilled in the art to make or use the embodiments of the disclosure and are not intended to limit the scope of the disclosure, which is defined by the claims.

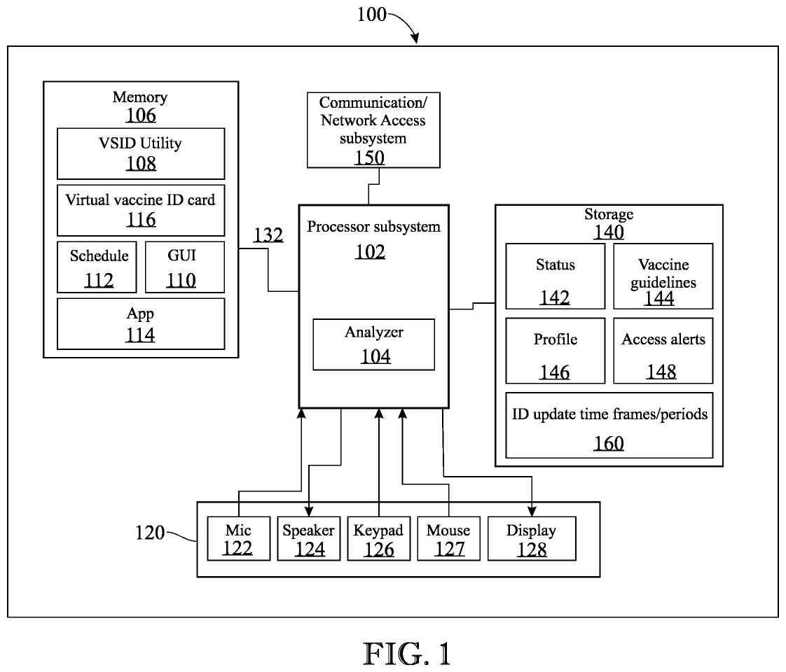

For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, the present disclosure is directed toward tracking received vaccinations and identifying an individual's vaccination status.

With reference now to the figures, and beginning with FIG. 1, there is depicted a block diagram representation of an example data processing system (DPS), as utilized within one embodiment. DPS may be a server, a digital audio workstation, a personal computer, a portable device, such as a personal digital assistant (PDA), a smart phone, and/or other types of electronic devices that may generally be considered processing devices or computing systems/devices. As illustrated, DPS 100 comprises at least one processor subsystem 102 connected to system memory 106 via system interlink/bus 132. DPS 100 executes one or more computer programs/applications to track vaccinations received by an individual and to identify a vaccination status of an individual based on received vaccinations in accordance with a vaccination schedule, according to the present disclosure.

In one or more embodiments, data processing device 100, which is managed by processor subsystem 102, also includes communication subsystem 150, data storage subsystem 140, and input/output (I/O) subsystem 120. As shown, processor subsystem 102 includes a shot analyzer module 104 to support the data analysis functionality of DPS 100. Processor subsystem 102 executes program code to provide operating functionality of data processing device 100. The software and/or firmware modules have varying functionality when their corresponding program code is executed by processor subsystem 102 or secondary processing devices (not explicitly shown) within DPS 100.

As illustrated, I/O subsystem 120 includes user interface devices including output devices such as audio output device(s)/speaker 124, and display device 128. In one or more implementations, display device 128 includes touch screen functionality enabling display device to function as both an input device and an output device. In addition, I/O subsystem 120 includes input devices including microphone 122, keypad 126 and mouse 127.

Processor subsystem 102 is communicatively coupled, via system bus/interlink 132, to device memory 106. In one or more embodiments, processor subsystem 102 is communicatively coupled via system interlink 132 to communication subsystem 150, data storage subsystem 140, and input/output subsystem 120. System interlinks 132 represents internal components that facilitate internal communication by way of one or more shared or dedicated internal communication links, such as internal serial or parallel buses. As utilized herein, the term “communicatively coupled” means that information signals are transmissible through various interconnections, including wired and/or wireless links, between the components.

Communication subsystem 150 may be configured to enable DPS 100 to communicate with a plurality of personal computing devices. The communication subsystem may include wired and/or wireless communication devices to facilitate networked communication. Communication subsystem 150 also includes a Network Access Module by which DPS 100 may connect to one or more access/external networks such as the Internet or wide area network (WAN), or an internal network such as an Ethernet (local area network LAN) or a Virtual Private Network (VPN).

In addition to the above-described hardware components of DPS 100, various features of the invention are completed/supported via software (or firmware) code or logic stored within memory 106 or other storage and executed by Processor subsystem 102. Thus, for example, illustrated within memory 106 are a number of software/firmware/logic components, including Application 114 and other applications (not shown). In addition, memory 106 comprises a vaccine status identification (VSID) module/logic/utility 108. Device memory 106 further includes an operating system (OS) (not shown), a firmware interface, such as basic input/output system (BIOS) or Uniform Extensible Firmware Interface (UEFI), and firmware (not shown). Device memory 106 includes a graphical user interface (GUI) 110, a vaccination schedule 112, a virtual vaccine ID card 116, and/or other computer data (not explicitly shown) used by the VSID utility 108 and/or the APP 114.

Data storage subsystem 140 enables further storage and retrieval of data, instructions, and code. In particular, data storage subsystem 140 provides applications, program code, and stored data on nonvolatile storage that is accessible by processor subsystem 102. For example, data storage subsystem 140 can provide, for use by the VSID utility 108, the vaccination status 142, the vaccine guidelines 144, individual profile/personal data 146, access alerts 148, and allowable ID card update time periods/frames 160. In addition, data storage subsystem 140 can provide a selection of program code and applications, and other related application(s) that can be used to support vaccination tracking and identification of an individual's vaccine status. These applications can be loaded into device memory 106 for execution by processor subsystem 102.

In actual implementation, the VSID logic 108 may be combined with the App 114 to provide a single executable component, collectively providing the various functions of each individual component when the corresponding combined component is activated. For simplicity, the VSID logic/utility 108 is illustrated and described as a stand-alone or separate logic/firmware component, which provides specific functions, as described below.

In one embodiment, DPS 100 communicates with a software deploying server (not shown) via a network (e.g., the Internet) using communication subsystem/network access module 150. Then, VSID utility 108 may be deployed from/on the network, via the software deploying server. With this configuration, the software deploying server performs all of the functions associated with the execution of VSID utility 108. Accordingly, DPS 100 is not required to utilize internal computing resources of DPS 100 to execute VSID utility 108.

The VSID utility 108 provides tracking of vaccinations, such as COVID-19 vaccinations, received by an individual. Based on the received vaccinations, the VSID utility 108 is able to determine the vaccine status of the individual based on a specified vaccination schedule. According to the aspect, a scannable card having vaccine data is designed to support access to the vaccine data using one or more of a QR code and a bar code. An authorized viewer of the vaccine data can be presented with audio, visual and/or textual content corresponding to the vaccine data. According to an aspect of the present disclosure, the VSID utility/module 108 enables an authorized individual to provide entry of vaccine related data via the GUI 110 corresponding to App 114. According to one or more aspects, the individual can be presented with aural content via the speakers 124. The individual may also be visually presented via the display device 128 with image, video and/or textual content. In addition, the individual may be visually presented with one or more associated selection buttons within the GUI 110. Using an input device, such as the mouse 127, the keypad 126 or the microphone 127, the participant can provide an input selection and/or vaccine-related data.

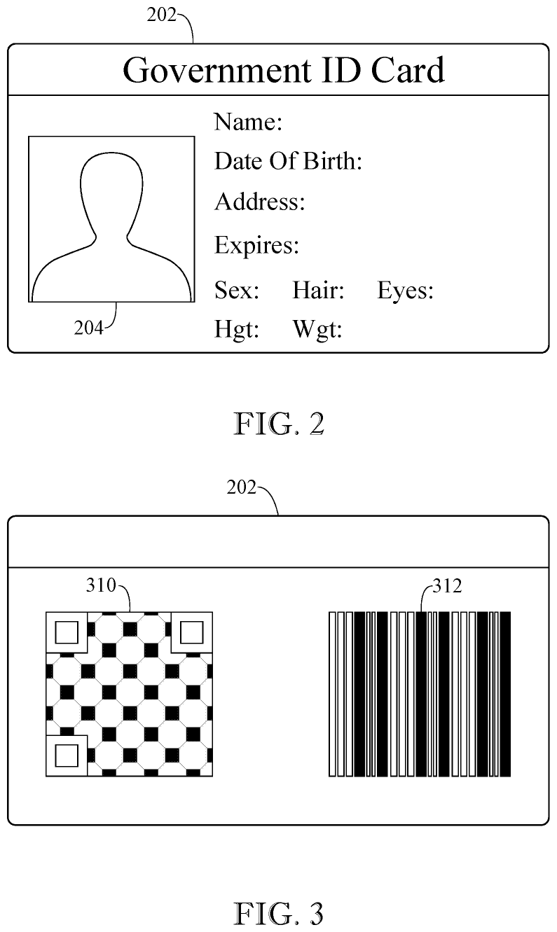

Certain of the functions supported and/or provided by the VSID utility/module 108 are implemented as processing logic (or code) executed by processor subsystem 102 and/or other device hardware, which processing logic enables the device to implement/perform those function(s). Among the software code/instructions/logic provided by the VSID module 108, and which are specific to the disclosure, are: (a) logic for establishing, using vaccination guidelines, a vaccination schedule for administering a specified vaccine via one or more vaccine injections/shots given to an individual; (b) logic for determining whether the individual receives one or more vaccine injections for the specified vaccine according to the vaccination schedule; (c) logic for determining a vaccine status of the individual based on the received vaccine injections and the vaccination schedule; (d) logic for integrating/uploading information associated with a vaccine status and a vaccination schedule into a secure readable/scannable vaccine identification card issuable to the individual; (e) logic for providing an authorized individual/viewer with secure access to vaccine information on the readable vaccine ID card; and (f) logic for providing an alert each time a unique code corresponding to the vaccine ID card is utilized. According to the illustrative embodiment, when Processor subsystem 102 executes the VSID logic/module 108, DPS 100 initiates a series of functional processes that enable the above functional features as well as additional features/functionality. These features/functionalities are described in greater detail below within the description of FIGS. 2-10.

Those of ordinary skill in the art will appreciate that the hardware components and basic configuration depicted in FIG. 1 may vary. The illustrative components within DPS 100 are not intended to be exhaustive, but rather are representative to highlight essential components that are utilized to implement the present disclosure. For example, other devices/components may be used in addition to or in place of the hardware depicted. The depicted example is not meant to imply architectural or other limitations with respect to the presently described embodiments and/or the general disclosure.

Referring now to FIG. 2, a front side of a government-issued vaccination ID card is illustrated, according to one or more embodiments of the disclosure. A vaccination ID card 202 includes a front side which shows a picture 204 of the individual to which the vaccination ID card 202 has been issued. In addition, other individual data, such as data of birth (DOB), address, and an expiration date of the vaccine ID card 202 is shown on the front side of the vaccine ID card. In addition, other identifying data for the individual, such as sex, weight, and height, are shown on the front side of the vaccine ID card 202.

FIG. 3 presents an illustration of a back side of a government-issued vaccination ID card, according to one or more embodiments of the disclosure. The vaccination ID card 202 includes a back side which shows a QR code 310 and a bar-code 312. According to an aspect, COVID-19 vaccine data is uploaded onto the scannable vaccine ID card 202 both with QR code 310 and bar code 312 accesses. According to an aspect, vaccine status and other vaccine related information is obtained by an authorized individual that scans the bar-code 312. According to an aspect, the VSID module 108 identifies a vaccination status as one of (i) unvaccinated, (ii) partially vaccinated and (iii) completely vaccinated. According to one or more related aspects, an unvaccinated status indicates that that an individual has not received any vaccination shots. A partially vaccinated status indicates that an individual has received either one or a portion of multiple vaccination shots, or that less than a threshold number of days has elapsed since the final shot of a single-shot vaccination program, or a multi-shot vaccination program was received. A “completely vaccinated” status indicates that the individual has received a final shot of vaccination program that requires one shot or multiple shots, and that a threshold number of days has elapsed since the final shot was received.

According to one or more aspects, the VSID module 108 enables an individual that scans the QR code to access a vaccination App 114 by which the individual may enter vaccine related information.

According to an aspect, once the vaccine ID card 202 is scanned, the authorized viewer will have temporary access to the card holder's vaccine data for viewing purposes only. In addition, the vaccine ID card 202 can be designed to include an encrypted device in order to prevent fraudulent access.

According to an aspect, the purpose of COVID-19 vaccine ID card 202 is to identify the cardholder as a vaccinated individual. As a result, the individual is determined to represent a low risk for transmitting a specific virus.

According to an aspect, the C-ID card or corresponding information is integrated with and/or linked to a government issued ID such as a driver's license or a state ID card.

According to another aspect, the C-ID card/document can be utilized within a fast pass mechanism/system. For example, the VSID module provides secure fast access/entrance to individuals traveling through airports, cruise ships, and large events, such as professional, sporting or entertainment events held within stadiums. According to an aspect, the VSID module provides an additional layer of security to a safety screening process.

According to an aspect, the VSID module is able to cross reference data with a government's Center for Disease Control and Prevention “CDC” database to prevent unauthorized data duplication.

According to an aspect, the vaccine ID card 202 is created to clearly identify that the card holder has either started the vaccination shot process by receiving a first dose of vaccine or has received both doses and/or has been fully vaccinated. The VSID module 108 provides multiple safety and security measures via a QR code, a bar-code, and a smart-phone App.

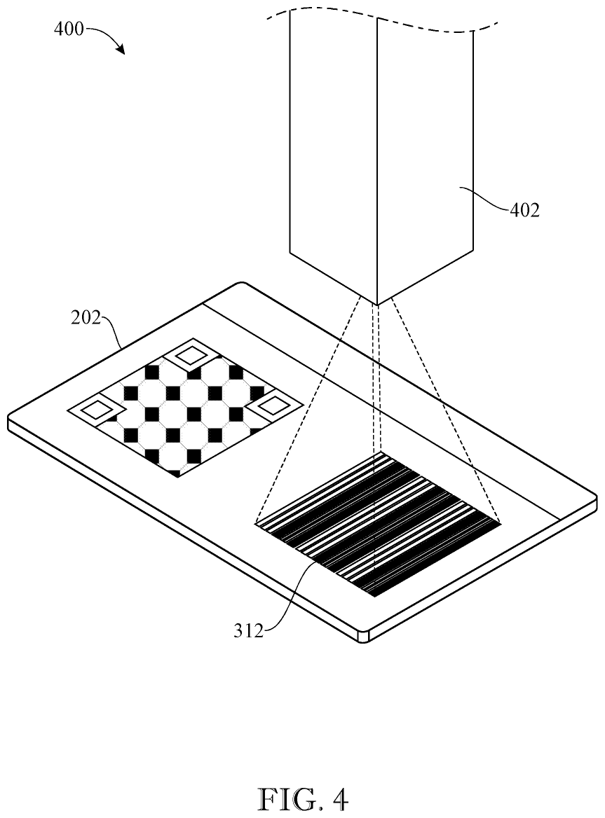

FIG. 4 illustrates a bar-code on a government-issued vaccination ID card being read by a scanner, according to one or more embodiments of the disclosure. Code reading environment 400 illustrates a portion of bar-code scanner 402 being used to scan a bar-code 312 of a vaccine ID card 202. According to an aspect, the VSID utility 108 enables a vaccine status to be obtained when an authorized individual scans the bar-code 312.

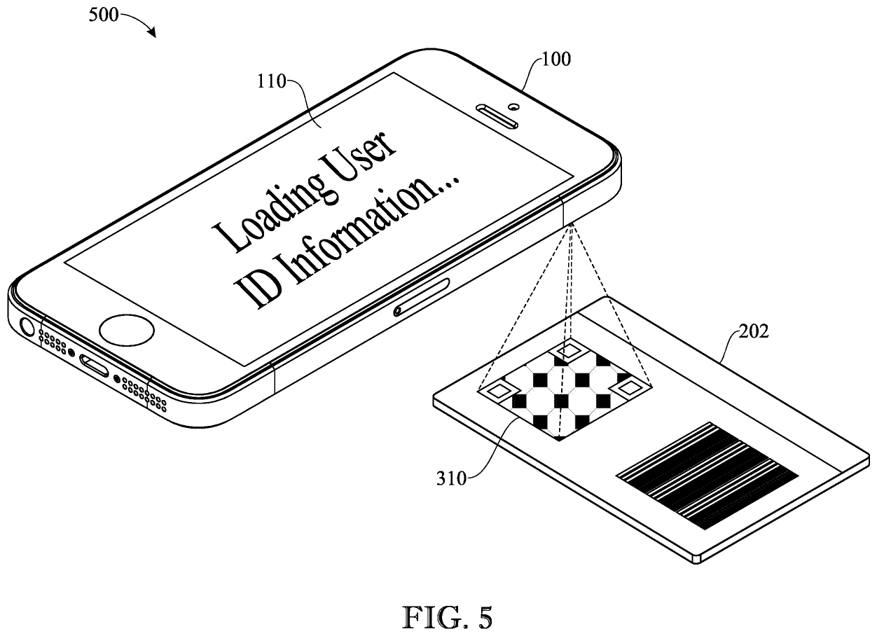

FIG. 5 illustrates a vaccination APP being accessed by reading a QR code on a government-issued vaccination ID card using a smart-phone camera, according to one or more embodiments of the disclosure. Code reading environment 500 illustrates a smart-phone 100 being used to read via a camera a QR code 310 of a vaccine ID card 202.

According to an aspect, the VSID utility 108 enables an authorized individual to scan the QR code 310 to access a vaccination App 114 and/or the individual's account via the vaccination App 114. As illustrated, the GUI 110 shows that the QR code scanning process has initiated a “loading of the user ID information”.

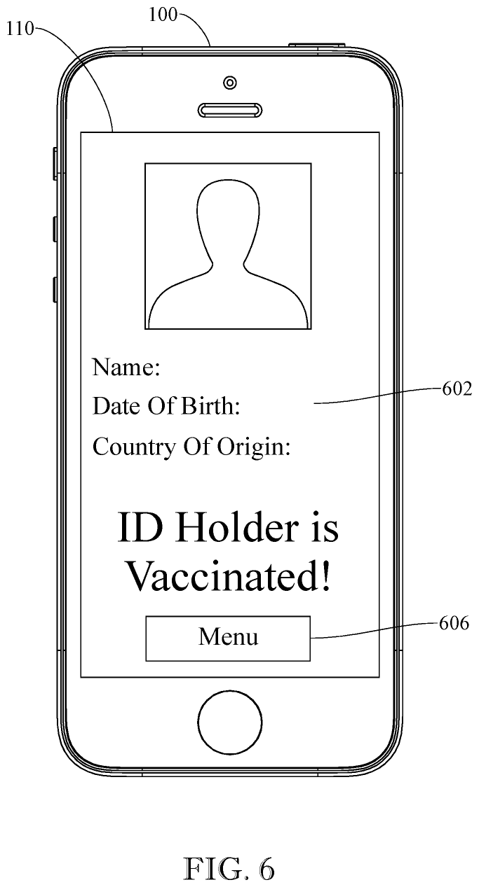

FIG. 6 illustrates a user interface providing a vaccination status of an individual, according to one or more embodiments of the disclosure. As illustrated, the GUI 110 identifies the cardholder by identification data 602, including picture, name, date of birth (DOB) and country of origin. In addition, the cardholder is shown to have a fully “vaccinated” status. Also illustrated is a menu button 606 to access a list of vaccination and/or cardholder related items.

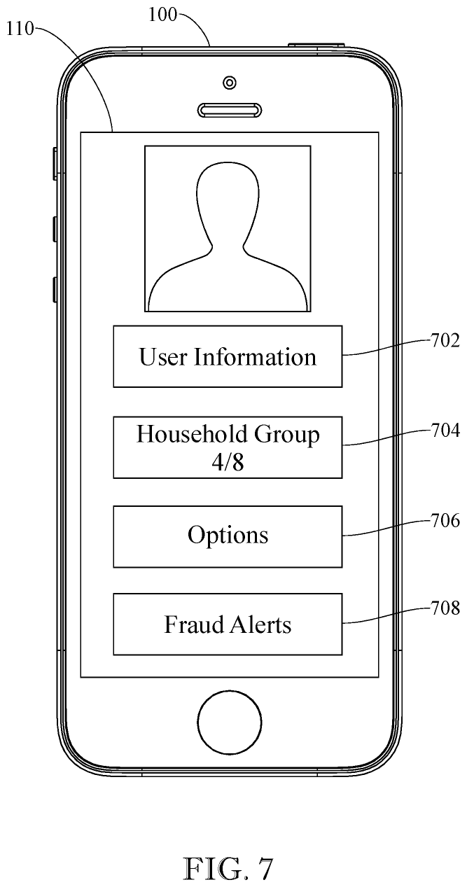

FIG. 7 illustrates a user interface showing a number of selectable menu buttons used to provide vaccine-related information, according to one or more embodiments of the disclosure. Following selection of the menu button 606 (FIG. 6), device 100 presents GUI 110 which shows a list of additional menu buttons including user information button 702, household group button 704, Options button 706 and Fraud alerts button 708.

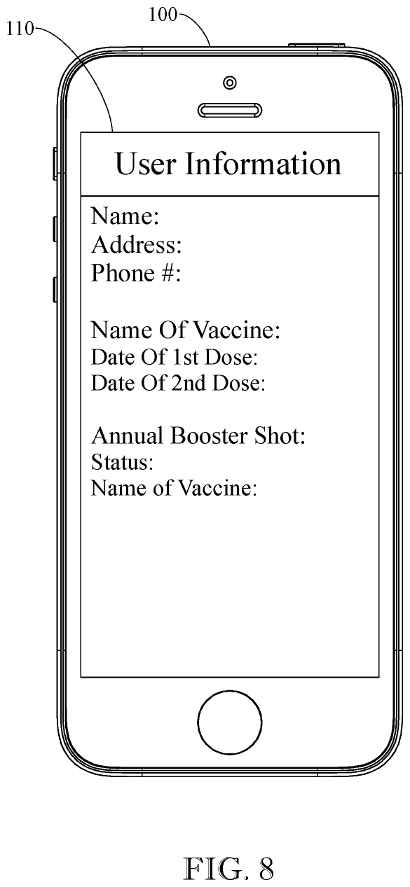

FIG. 8 illustrates a user interface showing a user information page for entry of vaccination data/schedule information, according to one or more embodiments of the disclosure. Following selection of the user information button 702 (FIG. 7), device 100 presents GUI 110 which shows a data entry page for user information. Included within the user information page are data entry fields for Name, Address, Phone number, Name of Vaccine, date of first dose, date of second dose, and annual booster shot date, status, and name. According to an aspect, a lot number for the vaccine to be used for verification purposes can also be entered in the user information page.

According to an aspect, when the cardholder and vaccine requestor is seeking a vaccination, information about vaccination required can be used to trigger a search for a nearest vaccine site location. According to an aspect, the VSID module 108 can inform the vaccine requestor of the name of the vaccine that is available at a given location. Thus, the requester can determine whether to opt out of receiving the available vaccine if the vaccine is not the first preference for the requester. As a result, the VSID module 108 can provide a list that includes alternative vaccine options and locations.

According to an aspect, if a vaccine ID card 202 is loss or misplaced the VSID module 108 can allow a new card to be issued once the requestor passes verification.

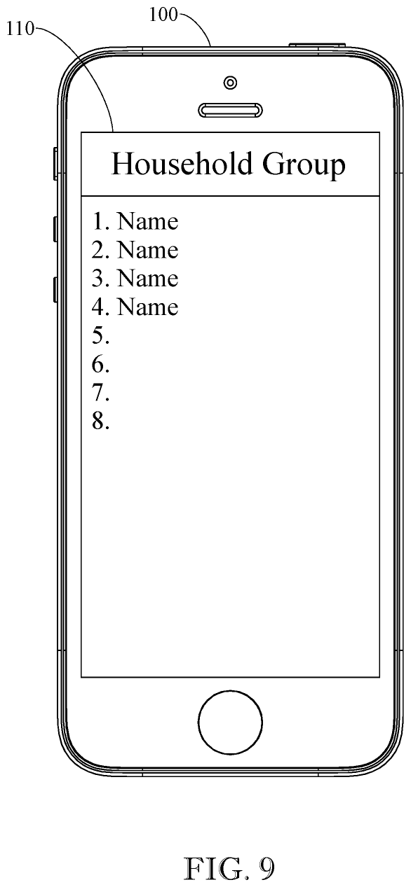

FIG. 9 illustrates a user interface providing a household group page for entering names of members to be included in the group, according to one or more embodiments of the disclosure. Following selection of the household group button 704 (FIG. 7), device 100 presents GUI 110 which shows a data entry page for the names of individuals to be added to the household group.

According to an aspect, a household group is established to enable fast and efficient access to vaccination data for the entire group. According to one or more related aspects, the household group is established to enable access to the entire household's vaccine data to facilitate access for travel related purposes for the entire group.

According to an aspect, the VSID module enables individuals that obtain the App to join or add themselves to the household group. According to an aspect, the VSID module may require that the individual submitting a request to be added to the household group use a verification code to confirm the request to be added to household group.

According to one or more related aspects, the VSID module enables the household group to receive access to travel related services/products at discounted/group rates.

According to one or more aspects, the VSID module can specify a limit on the number of individual members in a group and a number of associated groups per App. For example, the VSID module may specify a limit of eight (8) members in a group and a maximum of two associated groups per App. Thus, a maximum number of sixteen (16) members from a household can utilize a single App when split into two associated groups, each having a maximum of 8 members.

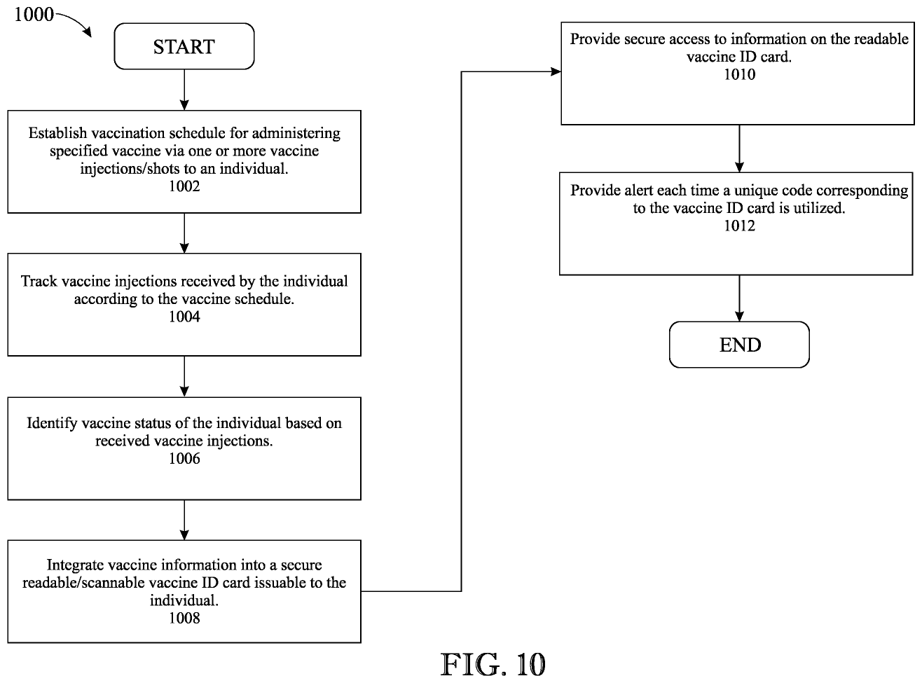

FIG. 10 is a flow chart illustrating a method by which the above process of the illustrative embodiments is completed. Although the method illustrated in FIG. 10 may be described with reference to components shown in FIGS. 1-9, it should be understood that this is merely for convenience and alternative components and/or configurations thereof can be employed when implementing the various methods. Key portions of the methods may be completed by the vaccine status identification (VSID) module 108 executing on processor subsystem 102 within DPS 100 (FIG. 1) and controlling specific operations of/on DPS 100, and the methods are thus described from the perspective of either/both the VSID module 108 and DPS 100 or other device that provides the functionality associated with one or more versions of the VSID module 108.

FIG. 10 is a flow chart illustrating the process of tracking received vaccinations and vaccination status of an individual. The process of FIG. 10 begins at the initiator/start block and proceeds to block 1002, at which the VSID module 108 establishes a vaccination schedule for administering a specified vaccine via one or more vaccine injections/shots to an individual. At block 1004, the VSID module 108 tracks vaccine injections received by the individual according to the vaccine schedule.

At block 1006, the VSID module 108 identifies the vaccine status of the individual based on the received vaccine injections. At block 1008, the VSID module 108 integrates vaccine information into a secure readable/scannable vaccine ID card issuable to the individual. According to an aspect, the VSID module 108 aggregates vaccine information, user information and other associated information as a virtual vaccine ID card 116 to be uploaded/integrated in a physical vaccine ID card.

At block 1010, the VSID module 108 provides secure access to information on the readable vaccine ID card. According to an implementation, the VSID module enables an authorized person to scan and read vaccine data using a bar-code on the back of the vaccine ID card. Once the vaccine ID card is scanned, the authorized viewer will have temporary access to the card holder data for viewing purpose only. According to an aspect, the vaccine ID card includes an encrypted device to prevent fraudulent data access.

At block 1012, the VSID module 108 provides an alert each time a unique code corresponding to the vaccine ID card is utilized. The process proceeds to the end block.

The method, the system and the computer program product track vaccinations received by an individual and provides proof of the individual's vaccination status. A vaccine status identification (VSID) module/utility establishes a vaccination schedule for administering a specified vaccine via one or more vaccine injections/shots to an individual. The VSID module determines whether the individual receives one or more vaccine injections for the specified vaccine according to the vaccination schedule. The VSID module determines a vaccine status of the individual based on received vaccine injections and the vaccination schedule. The VSID module integrates information associated with vaccination status and the vaccination schedule into a secure readable/scannable vaccine identification card issuable to the individual. The VSID module provides secure access to information on the readable vaccine ID card and provides an alert each time a unique code corresponding to the vaccine ID card is utilized.

According to one or more aspects, the VSID module is able to cross reference data with a government's Center for Disease Control and Prevention “CDC” database to prevent unauthorized data duplication. According to an aspect, the VSID module is able to update the card holder's vaccine information within specified time periods based on a schedule that must be followed in order to maintain a partial or complete vaccination status, according to vaccination guidelines and/or an established vaccination schedule. The VSID module provides the individual with an alert each time someone uses the individual's unique app code. According to one or more related aspects, the VSID module provides the alert as a fraud alert text.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalents.

In a first preferred embodiment of the present invention there is a method for providing a proof and a use of a first user's vaccination status comprising: (A) establishing a vaccination schedule and a date deadline for an administering of a vaccine to said first user; (B) verifying whether said first user has received said vaccine on or before said date deadline according to said vaccination schedule by a collection of a vaccine data from a vaccine provider; (C) determining a vaccine status of said first user from whether said first user has received said vaccine on or before said date deadline according to said vaccination schedule; (D) integrating an information associated with vaccine status and vaccine schedule into a secure readable/scannable vaccine identification card issuable to said first user; (E) providing a secure access to said information on said secure readable/scannable vaccine identification card; and (F) providing an alert each time a unique code corresponding to the vaccine ID card is utilized.

In an alternative embodiment of the first preferred embodiment of the present invention there is a method for providing a proof and a use of a first user's vaccination status further comprising including said first user in a household group having at least two users comprising a plurality of users including at least said first user and a second user before step (B).

In an alternative embodiment of the first preferred embodiment of the present invention there is a method for providing a proof and a use of a first user's vaccination status further comprising creating a vaccination data group for said household group and determining a group vaccine status for said household group as part of step (C).

In an alternative embodiment of the first preferred embodiment of the present invention there is a method for providing a proof and a use of a first user's vaccination status further comprising accessing an application and entering a name for each user on a data entry page for users to be added to said household group before step (B).

In an alternative embodiment of the first preferred embodiment of the present invention there is a method for providing a proof and a use of a first user's vaccination status further comprising each user in said plurality of users submitting a request to be added to said household group and using a verification code to confirm said request to be added to said household group.

To illustrate how this method extends beyond mere software, let's delve into the practical, technical, and operational aspects of each step and how they integrate with broader systems and real-world processes.

Monitoring outbreak exposure, separate from and in addition to user location, requires real time tracking systems to ensure effective management and prevention of disease spread. The present invention is directed to a method, a system, and a computer program product that tracks vaccinations received by an individual and provides proof of the individual's vaccination status. A vaccine status identification (VSID) module/utility establishes a vaccination schedule for administering a specified vaccine via one or more vaccine injections/shots to an individual. The VSID module determines whether the individual receives one or more vaccine injections for the specified vaccine according to the vaccination schedule. Based on the received vaccine injections and the established vaccination schedule, the VSID module determines the vaccine status of the individual. It integrates information associated with the vaccination status and the vaccination schedule into a secure, readable/scannable vaccine identification card issuable to the individual. Furthermore, the VSID module ensures secure access to information on the readable vaccine ID card and provides an alert each time a unique code corresponding to the vaccine ID card is utilized. This system enhances the accuracy and reliability of vaccination records, providing a critical tool for public health monitoring and individual compliance with vaccination protocols.

Establishing a vaccination schedule and deadline involves coordination with healthcare providers and possibly public health authorities to set up a vaccination schedule for users. This task requires healthcare coordination, engaging with healthcare providers to align on vaccination schedules and deadlines, which might involve manual and automated processes. Public health compliance ensures the schedule adheres to local or national public health guidelines, involving legal and regulatory considerations. Additionally, user communication informs users about their vaccination schedules through various channels (emails, SMS, physical mail), necessitating communication management beyond software.

Verification extends beyond software in several ways. Data collection involves the physical collection of vaccination data from healthcare providers, potentially requiring visits, secure data transfer protocols, and integration with electronic health record (EHR) systems. Authentication ensures the authenticity of the collected data through cross-checking with trusted sources, manual audits, or secure digital signatures. Compliance and privacy involve adhering to data privacy laws like HIPAA in the US, requiring legal expertise and secure data handling practices.

Determining vaccine status involves data analysis, either manual or automated, to determine compliance with the schedule. Medical oversight may require medical professionals to review the data to confirm the vaccine status, ensuring alignment with medical records. User notification involves informing users of their vaccine status through secure and reliable means, possibly including physical mail for those without digital access.

This integration is a complex process. Card design and manufacturing involve creating physical ID cards that are durable, secure, and user-friendly, selecting materials, incorporating security features (like holograms or embedded chips), and collaborating with manufacturers. Secure encoding uses methods to encode vaccine information onto the card, possibly involving cryptographic techniques and secure hardware. Distribution requires the physical distribution of ID cards to users, involving logistics, mailing systems, and possibly in-person distribution events.

Secure access requires robust infrastructure. Access technologies like QR codes, NFC, or smart cards allow secure access to vaccine information, involving both software and hardware components. Authorization systems ensure only authorized personnel can access the information, which may involve secure servers, authentication mechanisms, and possibly biometric verification. User training educates users and authorized personnel on how to use the ID cards and access systems securely.

The alert system encompasses several components. Monitoring infrastructure involves establishing infrastructure to monitor ID card usage in real-time, setting up servers, network components, and data processing systems. Alert mechanisms create methods to generate and send alerts, including SMS gateways, email servers, and push notification services. Security protocols ensure the alert system is secure and does not expose sensitive information, requiring cybersecurity measures and regular audits.

Creating household groups involves data management to manage relationships and data for multiple users within a group, ensuring data integrity and privacy. User interfaces design user-friendly interfaces for adding and managing household groups, involving user experience (UX) design and front-end development. Community outreach potentially engages with community organizations to help users form household groups, especially in underserved areas.

Group-level vaccination status requires data aggregation to aggregate individual vaccination data and determine a collective status, involving complex data processing and analysis. Reporting generates reports or dashboards for household groups to easily view their collective status, involving data visualization tools.

Data entry processes require user support, providing support channels (e.g., help-desks, tutorials) to assist users in entering their data correctly. Accessibility considerations ensure the data entry process is accessible to users with disabilities, involving compliance with accessibility standards.

Verification processes include workflow design for handling user requests and verification, involving both automated and manual steps. Security measures implement protection against fraudulent requests, including secure code generation and validation processes. User communication involves communicating with users throughout the verification process, ensuring they are informed and supported.

Collaboration with authorities involves working with public health authorities, healthcare providers, and possibly employers to implement and maintain the vaccination verification system. Public health impact contributes to broader public health efforts by ensuring accurate vaccination tracking and reporting, potentially aiding in disease prevention and control. Logistical coordination manages the logistics of card distribution, user support, and data collection, requiring coordination beyond digital systems.

In summary, the method described involves a blend of digital and physical processes, requiring collaboration with various stakeholders, adherence to legal and regulatory standards, and the integration of both software and hardware components. The system issues alerts and activates tracking with regard to user location for situations of criminal cases or missing persons, provides Travel IDs for households, reports infectious disease exposures, traces origins, alerts at-risk individuals, suggests remedies, and autonomously processes data for safety and health. This enhances abduction recovery and disease management during travel, such as on cruise ships. By ensuring a robust, secure, and effective approach, the method manages and verifies vaccination statuses comprehensively.

The method described for providing proof of a user's vaccination status has several key components, each contributing to a robust and secure system for verifying and managing vaccination information. Here's a breakdown of the “something more” in this method:

Establishing a vaccination schedule and deadline involves setting up a schedule for when the vaccine should be administered and a deadline by which it should be done. This ensures that the vaccination process is organized and time-bound.

Verifying vaccination status includes a verification process to check if the user has received the vaccine by the set deadline. This is done by collecting vaccine data from the provider, ensuring accuracy and reliability of the vaccination status.

Determining vaccine status is based on the collected data. The system determines whether the user has met the vaccination requirements according to the established schedule. This is crucial for maintaining accurate records of who is vaccinated.

Integration into a secure vaccine identification card involves incorporating the vaccination status and schedule information into a secure, readable, and scannable vaccine identification card. This card serves as a portable and easily accessible proof of vaccination.

Providing secure access ensures that access to the information on the vaccine ID card is secure. This protects the user's personal health information from unauthorized access.

An alert system is in place to notify whenever the unique code corresponding to the vaccine ID card is utilized. This could help in tracking usage, preventing fraud, and ensuring the card is used appropriately.

The combination of these steps creates a comprehensive and secure method for providing and verifying proof of vaccination, making it more than just a simple record-keeping system. It integrates verification, security, and real-time alert features to enhance reliability and trustworthiness.

In addition, the complexity of sharing approximate real-time locations of disease outbreaks and the vaccination status of users anonymously presents significant challenges. However, the system's ability to monitor user location, health status, and the risk of surrounding infections addresses these challenges by autonomously collecting and analyzing data from various sources. By comparing the user's health status and location data against current epidemiological information, the system can provide real-time, personalized risk assessments and recommendations for treatment. This approach not only aids in individual health management but also contributes to public health efforts by anonymously sharing crucial data to track and control disease outbreaks, thereby suggesting effective remedies and preventive measures.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A method for providing a proof and a use of a first user's vaccination status comprising:
(A) establishing a vaccination schedule and a date deadline for an administering of a vaccine to said first user;
(B) verifying whether said first user has received said vaccine on or before said date deadline according to said vaccination schedule by a collection of a vaccine data from a vaccine provider;
(C) determining a vaccine status of said first user from whether said first user has received said vaccine on or before said date deadline according to said vaccination schedule;
(D) integrating an information associated with vaccine status and vaccine schedule into a secure readable/scannable vaccine identification card issuable to said first user;
(E) providing a secure access to said information on said secure readable/scannable vaccine identification card; and
(F) providing an alert each time a unique code corresponding to the vaccine ID card is utilized.

2. The method of claim 1 further comprising including said first user in a household group having at least two users comprising a plurality of users including at least said first user and a second user before step (B).

3. The method of claim 2 further comprising creating a vaccination data group for said household group and determining a group vaccine status for said household group as part of step (C).

4. The method of claim 2 further comprising accessing an application and entering a name for each user on a data entry page for users to be added to said household group before step (B).

5. The method of claim 2 further comprising each user in said plurality of users submitting a request to be added to said household group and using a verification code to confirm said request to be added to said household group.
